(19) United States
(12) Patent Application Publication
(10) Pub. No.: US 2009/0035370 A1
(43) Pub. Date: Feb. 5, 2009

(54) DOSAGE FORM AND METHOD OF USE

(75) Inventors: Jonathan David Bortz, Saint Louis, MO (US); Michael Norman Grimshaw, Saint Louis, MO (US)

Correspondence Address: KV PHARMACEUTICAL COMPANY, 4080B WEDGEWAY COURT, EARTH CITY, MO, 63045, US

(73) Assignee: DRUGTECH CORPORATION, Wilmington, DE (US)

(21) Appl. No.: 12/183,989

(22) Filed: Jul. 31, 2008

Related U.S. Application Data
(60) Provisional application No. 60/953,641, filed on Aug. 2, 2007.

(51) Int. Cl. A61K 9/52; A61K 31/554; A61K 9/22; A61P 25/00

(52) U.S. Cl. 424/457; 514/211.13; 424/468

(57) ABSTRACT

A method for treating a medical condition for which quetiapine is indicated in a subject comprises comprising orally administering to the subject quetiapine or a pharmaceutically acceptable salt thereof in a daily dosage amount effective to treat said condition; wherein the quetiapine or salt thereof is administered in one to a plurality of dosage forms collectively comprising (a) a major quetiapine component in immediate-release form in a sedative effective amount, administered not earlier than about 3 hours prior to the start of a sleep period; and (b) either (i) a minor quetiapine component in extended-release, delayed extended-release or delayed pulsed-release form, wherein time of administration and release properties of the minor component provide substantial onset of release of quetiapine therefrom not earlier than about 6 hours after the start of the sleep period, or (ii) a plurality of minor quetiapine components in immediate-release form, administered sequentially during a waking period following the sleep period but not earlier than about 6 hours after the start of the sleep period; said minor component or components collectively being administered in an anxiolytic effective amount insufficient to cause an unacceptable degree of sedation. A dosage form suitable for use in such a method is also provided.

This application claims benefit of U.S. provisional application 60/953,641 filed on Aug. 2, 2007, the entirety of which is incorporated herein by reference.

### FIELD OF THE INVENTION

Described herein are pharmaceutical dosage forms comprising quetiapine and methods of use thereof, for example in treatment of conditions for which quetiapine is indicated.

### BACKGROUND OF THE INVENTION

Quetiapine, 1-[2-(4-dibenzo[b,f][1,4]thiazepin-11-yl-1-piperazinyl)ethoxy]ethanol, corresponds in structure to Formula (I) below:

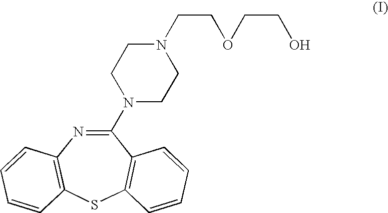

Quetiapine, including its fumarate salt, is a well known drug effective for treatment of psychiatric conditions. Quetiapine belongs to a group of drugs known as “atypical” or second-generation antipsychotics which have become increasingly popular alternatives to “typical” antipsychotics. Quetiapine fumarate is currently marketed by AstraZeneca under the brand name SEROQUEL® and has been approved by the United States Food and Drug Administration (FDA) for treatment of schizophrenia, bipolar mania and depressive episodes associated with bipolar disorder.

When administered orally, quetiapine is rapidly absorbed and generally reaches a peak plasma level within about 1.5 hours. Quetiapine is highly metabolized, with elimination occurring mainly through hepatic metabolism in the liver. The plasma half-life of quetiapine is typically about 5 to about 7 hours.

Quetiapine is an antagonist at several neurotransmitter receptors in the brain: serotonin 5HT₁ₐ and 5HT₂ receptors (IC₅₀=717 and 148 μM respectively), dopamine D₁ and D₂ receptors (IC₅₀=1268 and 329 nM respectively), histamine H₁ receptor (IC₅₀=30 nM), and adrenergic a 1 and a 2 receptors (IC₅₀=94 and 271 nM respectively). See Physicians' Desk Reference (PDR) 61st ed. (2007), pp. 690-695. Thomson PDR, Montvale, N.J. The mechanism of action of quetiapine, as with that of other antipsychotic agents, is unknown.

Gefvert et al. (1998) Psychopharmacology 135:119-126 suggested that the antipsychotic effect of quetiapine is thought to be mediated through antagonist activity at dopamine and serotonin receptors, specifically, the D₂ dopamine and 5-HT₂ serotonin receptor subtypes.

Kapur & Seeman (2001) Am. J. Psychiatry 158(3):360-369 proposed that fast dissociation from the D₂ receptor makes an antipsychotic more accommodating of physiological dopamine transmission, permitting an antipsychotic effect without motor side effects, prolactin elevation, or secondary negative symptoms. They stated that because all antipsychotics attach to the D₂ receptor with a similar rate constant and differ only in how fast they come off of the receptor, the degree of D₂ receptor affinity is thought to serve as a predictor of adverse side effects. They reported that recent studies showed that although patients receiving 300-600 mg/day quetiapine showed less than 20% D₂ occupancy at 12 hours after receiving a dose, at 2 hours they showed occupancies in the 60% range. Theoretically, it was proposed that this should allow for normal physiological surges of dopamine to elicit their normal effects, thus minimizing the risk of side effects such as pseudo-parkinsonism and elevations in prolactin.

Kapur et al. (2000) Archives of General Psychiatry 57:553-559 measured D₂ and 5-HT₂ₐ occupancy using positron emission tomography (PET) imaging. Quetiapine was found to be an effective antipsychotic and to improve extrapyramidal symptoms (EPS) and prolactin level elevation noted at baseline. It reportedly achieved these results with minimal (0-27%) D₂ occupancy 12 hours after the last dose. Study of additional subjects revealed that quetiapine does give rise to transiently high (58-62%) D₂ occupancy 2 to 3 hours after a single dose and that occupancy then decreases to minimal levels in 12 hours.

Tauscher-Wisniewski et al. (2002) J. Clin. Psychiatry 63(11):992-997 proposed that transiently high dopamine D₂ receptor occupancy by antipsychotic medication may be sufficient to induce an antipsychotic response. They reported results showing that patients with a first episode of schizophrenia responded to treatment with a single daily dose of quetiapine despite only transiently high D₂ receptor occupancy and questioned whether continuously high D₂ blockade is necessary for obtaining an antipsychotic response.

Farah (2005) Prim. Care Companion J. Clin. Psychiatry 7(6):268-274 reported antipsychotic effects through use of quetiapine with rapid but transient binding (58-64% D₂ occupancy at 2-3 hours after a dose, later declining to 0-27% occupancy by the end of a 12-hour dosing interval). Such transient binding appears to be sufficient for the clinical efficacy of quetiapine, while never approaching the 78% threshold of D₂ occupancy for EPS side effects. Farah reported data indicating that thresholds of 65% and 78% D₂ receptor occupancy may be associated with clinical efficacy and EPS respectively. It has been proposed that the atypicality of quetiapine may be explained by its ability to transiently occupy D₂ receptors.

Horacek et al (2006) CNS Drugs 20(5):389-409 reported that transient D₂ receptor antagonism is sufficient to obtain an antipsychotic effect, while permanent D₂ receptor antagonism increases the risk of adverse effects such as EPS.

The most common side effect of quetiapine is sedation. Quetiapine's antagonistic effect on the histamine H₁ receptor is thought to be responsible for its sedative effects. It is believed that this common side effect has led to off-label prescription of quetiapine fumarate for sleep and anxiety disorders.

While the sedative effects may be useful in treatment of sleep disorders, daytime sedation is a detrimental side effect that for many prevents use of the drug. Therefore, attempts have been made to balance the antipsychotic and sedative effects of quetiapine to provide a therapeutically effective regimen. Specifically, a dosing regimen has been mentioned wherein “[u]sers typically take smaller doses during the day for the neuroleptic properties and [a] larger dose at bedtime for the sedative effects, or divided in two equally high doses every 12 hours.” See Wikipedia (http://en.wikipedia.org/wiki/antipsychotic).

Miller (2004) Prim. Care Companion J. Clin. Psychiatry 6(suppl 2):3-7 proposed instructing a patient bothered by daytime sedation to take his or her medication at bedtime or to take the majority of the dose at night.

U.S. Patent Application Publication No. 2005/0158383 of Boehm & Dundon proposes various quetiapine formulations having a variety of release (including immediate, sustained, delayed and pulsed release) and pharmacokinetic profiles.

A need remains in the art for an improved regimen for administration of quetiapine to take best advantage of its antipsychotic, antidepressant and sedative properties, and particularly for an improved dosage form that provides such a regimen by once-daily administration.

### SUMMARY OF THE INVENTION

There is now provided a method for treating a medical condition for which quetiapine is indicated in a subject, comprising administering to the subject quetiapine or a pharmaceutically acceptable salt thereof in a daily dosage amount effective to treat such a condition; wherein the quetiapine or salt thereof is administered in one to a plurality of dosage forms collectively comprising
(a) a major quetiapine component in immediate-release form in a sedative effective amount, administered not earlier than about 4 hours prior to the start of a sleep period; and
(b) either (i) a minor quetiapine component in extended-release, delayed extended-release or delayed pulsed-release form, wherein time of administration and release properties of the minor component provide substantial onset of release of quetiapine therefrom not earlier than about 6 hours after the start of the sleep period, or (ii) a plurality of minor quetiapine components in immediate-release form, administered sequentially during a waking period following the sleep period but not earlier than about 6 hours after the start of the sleep period; such minor component or components collectively being administered in an anxiolytic effective amount insufficient to cause an unacceptable degree of sedation.

In one embodiment the major and minor quetiapine components are administered not earlier than about 4 hours prior to the sleep period in one to a plurality of dosage forms. Each such dosage form comprises both the major component and, in delayed extended-release or delayed pulsed-release form, the one or more minor components. Such a dosage form may be, for example, an oral dosage form. Upon exposure of any one such oral dosage form to gastrointestinal (GI) fluid or an in vitro medium that simulates GI fluid, substantial onset of release of quetiapine from the one or more minor components exhibits a delay of at least about 6 hours after initiation of said exposure.

There is further provided a pharmaceutical dosage form useful in the above method.

There is still further provided an orally deliverable pharmaceutical dosage form comprising quetiapine in a dosage amount effective for treatment of a condition for which quetiapine is indicated, and at least one pharmaceutically acceptable excipient; the quetiapine being in a form of free base and/or a pharmaceutically acceptable salt thereof, and comprising
(a) a major component in immediate-release form in a sedative effective amount, and
(b) one or more minor components in delayed extended-release or delayed pulsed-release form, collectively in an anxiolytic effective amount insufficient to cause an unacceptable degree of sedation;
   wherein, upon exposure of the dosage form to GI fluid or an in vitro medium that simulates GI fluid, substantial onset of release of quetiapine from the one or more minor components exhibits a delay of about 6 to about 24 hours after initiation of said exposure.

In one embodiment, when one to a plurality of such dosage forms are administered orally once daily to a subject, the major component provides greater than about 50% occupancy by quetiapine of striatal dopamine D₂ receptors within about 4 hours after administration, such occupancy subsequently falling below about 50%; and the one or more minor components upon release thereof are collectively ineffective to restore such occupancy to a level above about 50%.

There is still further provided an orally deliverable pharmaceutical dosage form comprising quetiapine, in a form of free base and/or a pharmaceutically acceptable salt thereof, and at least one pharmaceutically acceptable excipient; the dosage form upon once-daily administration exhibiting a steady-state pharmacokinetic (PK) profile having at least two peaks, Cₘₐₓ₁ and Cₘₐₓ₂, and a trough, Cₘᵢₙ₁, therebetween; wherein
(a) Cₘₐₓ₁ is a transient peak occurring less than about 4 hours after administration and is sufficient to initiate occupancy by quetiapine of striatal dopamine D₂ receptors at a level greater than about 50%, which level subsequently falls below about 50%;
(b) Cₘᵢₙ₁ occurs between about 6 hours and about 18 hours after administration;
(c) the ratio of Cₘₐₓ₁ to Cₘᵢₙ₁ is at least about 2:1; and
(d) the ratio of Cₘₐₓ₁ to Cₘₐₓ₂ is at least about 1.5:1.

Other embodiments will be evident from the detailed description that follows.

### BRIEF DESCRIPTION OF THE DRAWINGS

FIG. 1 is a diagrammatic representation of the PK profile of an illustrative once-daily dosage form of a first embodiment described herein having a major quetiapine component in immediate-release form and a minor quetiapine component in delayed extended-release form.

FIG. 2 is a diagrammatic representation of the PK profile of an illustrative once-daily dosage form of a second embodiment described herein having a major quetiapine component in immediate-release form and a plurality of minor quetiapine components in delayed pulsed-release form.

FIG. 3, included for comparative purposes, is a diagrammatic representation of the PK profile of a once-daily dosage form having a major quetiapine component in immediate-release form and a minor quetiapine component in “delayed immediate-release” form.

FIG. 4, included for comparative purposes, is a diagrammatic representation of the PK profile of a once-daily extended-release quetiapine formulation.

### DETAILED DESCRIPTION

In one embodiment described herein, a method is provided for treating a medical condition for which quetiapine is indicated. Quetiapine has been found useful in treatment of a wide range of diseases and disorders, not all of which are known to be mediated by dopamine receptors. Quetiapine is approved in the U.S. for treatment of schizophrenia, bipolar mania and bipolar depression. AstraZeneca is reportedly conducting a number of clinical trials and ongoing studies to test efficacy of SEROQUEL® and SEROQUEL XR™ quetiapine fumarate for various conditions (http://www.astrazenecaclinicaltrials.com). No admission is made as to the status of this report as prior art or otherwise with respect to the present invention.

Conditions for which quetiapine is “indicated” herein are not restricted to those for which U.S. FDA approval has been sought or granted. Quetiapine is indicated for treatment of a variety of psychiatric conditions and/or sleep disorders.

In some embodiments, the condition for which quetiapine is indicated is a psychiatric condition. The term “psychiatric condition” herein refers to any of various conditions characterized by impairment or disturbance of an individual's normal cognitive, emotional or behavioral functioning. Illustratively, psychiatric conditions that may be treatable with quetiapine include schizophrenia, bipolar mania, depressive episodes associated with bipolar disorder, post-traumatic stress disorder, obsessive-compulsive disorder, borderline personality disorder, major depressive disorder, mood and anxiety disorders, autism, alcoholism, dementia-related psychosis, hallucinations in Parkinson's disease patients using ropinirole, Tourette's syndrome, aggression and intermittent explosive disorder in fragile X syndrome patients, and co-morbid substance abuse.

In other embodiments, the condition for which quetiapine is indicated is a sleep disorder. The term “sleep disorder” herein refers to any disturbance of usual sleep patterns or behaviors. Illustratively, sleep disorders that may be treatable with quetiapine include insomnia, sleepiness during the day, restless legs syndrome, nightmares, and sleep apnea. Sleep and psychiatric disorders such as schizophrenia and depression are closely related. Psychiatric disorders are the leading cause of insomnia. On the other hand, lack of sleep may cause psychiatric problems such as paranoia and hallucinations. Further, sleep difficulties may make a psychiatric disorder worse.

A “sleep period” herein means a major sleep period, for example as dictated by a subject's circadian rhythm of sleep and wakefulness, and may occur at any time of day, but for most subjects is a night-time period.

The terms “treat”, “treating”, “treatment” and “treatable” herein will be understood, except where the context demands otherwise, to include palliative use, i.e., act to reduce, alleviate, or relieve symptoms of an underlying cause of a disease or disorder, and/or use to address an underlying cause of a disease or disorder.

The subject having a condition for which quetiapine is indicated, and treatable by the present method, may be a human patient of either sex and of any age, or may be a nonhuman animal, particularly a domestic or companion animal, illustratively a cat, dog or horse.

The total amount of quetiapine administered in a 24-hour period according to the present method is an amount that is therapeutically effective for the particular condition to be treated.

Illustratively, for management of schizophrenia and/or psychosis associated therewith, a suitable daily dose of quetiapine for an adult human subject will generally be about 300 to about 400 mg; for management of bipolar mania, a suitable daily dose will generally be about 400 to about 800 mg; for management of depressive episodes of bipolar disorder, a suitable daily dose will generally be about 150 to about 600 mg; and for treatment of a sleep disorder, a suitable daily dose will generally be about 50 to about 300 mg. Doses lower than these ranges may be administered at the beginning of a treatment period, and doses higher than these ranges may be found necessary or desirable for particular patients.

In one embodiment, the condition to be treated is one that is mediated at least in part by dopamine receptors, more particularly the dopamine D₂ receptor. Without being bound by theory, it is believed that a useful antipsychotic response to quetiapine therapy is obtained when peak D₂ receptor occupancy by quetiapine in a 24-hour period is at least about 50%, and that such occupancy may be achieved by a dose that is sufficient to provide a transient peak (Cₘₐₓ) quetiapine concentration in plasma of at least about 200 ng/ml, for example at least about 250 ng/ml or at least about 300 ng/ml. In some situations a higher peak occupancy, for example at least about 55%, at least about 60% or at least about 65%, may be desirable; such occupancy levels may require a higher Cₘₐₓ of, for example, at least about 350 ng/ml, at least about 400 ng/ml or at least about 450 ng/ml.

It should be noted, again without being bound by theory, that initial occupation of D₂ receptors by quetiapine is driven by Cₘₐₓ, but that de-occupation occurs slowly, so that the level of occupancy remains high for some time, for example up to about 10 hours, after plasma concentration of quetiapine has declined from the Cₘₐₓ. Furthermore, again without being bound by theory, it is believed that the antipsychotic benefits of quetiapine outlast the period of high D₂ receptor occupancy.

Thus the present method relies for its antipsychotic effect on administering a major quetiapine component once daily, in an immediate-release form. It is believed that this provides a transient Cₘₐₓ that, in turn, results in a level of D₂ receptor occupancy high enough to provide a desired antipsychotic effect, which is then maintained for at least a substantial portion of a 24-hour period after plasma quetiapine levels have passed their peak and even after D₂ receptor occupancy has fallen below about 50%. The intermittent nature of D₂ receptor occupancy provided by the present method is believed to result in a lower incidence of EPS and prolactin elevation, i.e., loss of atypicality, than would occur following administration of an extended-release quetiapine dosage form at a dose providing more or less continuous D₂ receptor occupancy.

As indicated hereinabove, quetiapine exhibits affinity for a number of receptors in the brain other than the dopamine D₂ receptor, in some cases with a lower IC₅₀ than in the case of D₂. Occupancy of these receptors is not necessarily as protracted as D₂ receptor occupancy; thus effects mediated by these receptors, whether adverse or beneficial, tend to be more transient than the antipsychotic effect.

The most common side effect of quetiapine is somnolence or sedation, which is believed to be mediated at least in part by interaction of quetiapine with the histamine H₁ receptor. According to the present method, a major quetiapine component is released in the GI tract prior to or in the early part of a sleeping period, for example in the evening or at night. Such a method takes advantage of the sedative “side effect” by maximizing sedation at a time when the patient is trying to fall asleep or is already sleeping. Furthermore, such a method minimizes unwanted sedation during a waking period, for example daytime sedation, by providing the major quetiapine component in immediate-release form, permitting substantial clearance of quetiapine by the end of the sleep period.

Because psychiatric disorders are a leading cause of insomnia, the present method offers great benefit to psychiatric patients who experience sleep disturbances. More specifically, disturbed sleep is prevalent among patients with schizophrenia (Cohrs et al. (2004) Psychopharmacology 174:421-429. Therefore, note should be taken that in the context of the embodiments described herein, sedation within about 2 hours prior to or during a sleep period is not considered an unwanted side effect.

At very low doses, quetiapine may provide an anxiolytic or “calming” effect that may or may not be mediated by interaction with the histamine H₁ receptor. That quetiapine-induced calming may occur without excessive sedation may reflect a difference in H₁ receptor occupancy at different dosage levels, or involvement of different receptors, or a combination thereof.

The minor quetiapine component delivered according to the embodiments described herein provides a supplement or boost to plasma level that is sufficient to elicit the desired anxiolytic effect but insufficient to elicit a sedative effect at an unacceptable level. Individual patients differ in their response to quetiapine and/or their tolerance of sedation, thus the threshold plasma level (and therefore quetiapine dose delivered by the one or more minor components) that leads to an unacceptable degree of sedation may vary considerably among patients. A suitable dose may, however, readily be determined by the skilled physician without undue experimentation.

It is believed, without being bound by theory, that an anxiolytic effective amount insufficient to cause unacceptable sedation will generally not be sufficient to restore striatal dopamine D₂ receptor occupancy by quetiapine to a level greater than about 35%, or greater than about 50%. Thus, even with administration or release of the minor quetiapine component as provided herein, re-occupation of D₂ receptors at a level greater than about 35%, or greater than about 50%, is believed not to occur until administration of the next daily major component dose.

Release of the minor component does not occur as a single “burst” but as a plurality of “bursts” or a sustained release over a period of several hours. The resulting anxiolytic or calming effect lasts for a substantial part of the waking period, for example in various embodiments at least about 6 hours, at least about 8 hours, at least about 10 hours, at least about 12 hours or for substantially the whole of the waking period.

Thus the present method is believed to provide at least the following benefits:
(a) a “bolus” dose of quetiapine sufficient to populate striatal dopamine D₂ receptors at an antipsychotic effective level;
(b) decline of D₂ receptor occupancy prior to the next “bolus” dose to a level and for a period sufficient to minimize loss of atypicality of quetiapine;
(c) sedation during at least part of the sleep period, such sedation wearing off by the end of the sleep period; and
(e) a calming effect during a substantial part of the waking period, not accompanied by excessive sedation or excessive repopulation of D₂ receptors.

Quetiapine may be administered as free base or as a pharmaceutically acceptable salt thereof. Salts include hydrochloride, hydrobromide, sulfate, sulfamate, phosphate, nitrate, acetate, trifluoroacetate, propionate, oxalate, malonate, succinate, glutarate, glycolate, stearate, lactate, malate, tartrate, citrate, ascorbate, pamoate, maleate, hydroxymaleate, fumarate, phenylacetate, arginate, asparginate, glutamate, benzoate, salicylate, mesylate, esylate, besylate, sulfanilate, 2-acetoxybenzoate, toluenesulfonate, methanesulfonate, ethanedisulfonate, oxalate and isethionate salts and combinations thereof. Salts may be partial, having a base to anion mole ratio of greater than 1:1, for example about 2:1 as in the case of a hemi-salt. The “fumarate salt” of quetiapine as referred to in much of the literature is a 2:1 salt and is sometimes called the hemifumarate salt.

The present method comprises administering daily to the subject one to a plurality of dosage forms that, following administration, collectively release a major and one or more minor quetiapine components. Reference herein to “quetiapine” will be understood to embrace quetiapine free base and pharmaceutically acceptable salts such as fumarate salts of quetiapine, unless the context demands otherwise. Amounts and doses of quetiapine or its salts are expressed on a quetiapine free base equivalent basis unless expressly indicated otherwise.

The major and minor components of quetiapine may be administered by any suitable route or routes of administration and in dosage forms appropriate to such routes. While the embodiments described herein make reference to oral administration and orally deliverable dosage forms that release quetiapine in the GI tract of the subject, the present invention is not limited thereto.

In the description that follows, the major component is stated to provide a “sedative effective” amount of quetiapine and the one or more minor components an “anxiolytic effective” amount of quetiapine. These properties are not to be read as limiting in any way the psychiatric conditions and sleep disorders treatable by methods and compositions described herein. Thus, for example, quetiapine administration according methods described herein may address a psychotic condition such as schizophrenia or bipolar disorder, even though the major and minor components of quetiapine administered are respectively defined herein in terms of their sedative and anxiolytic effects.

A “major” component herein comprises more than 50% of the total daily dose of quetiapine administered. Thus the one or more “minor” components collectively comprise less than 50% of the total daily dose of quetiapine administered.

Dealing first with the major quetiapine component, this component is in immediate-release form and is administered in a sedative effective amount. Time of administration is such that substantial onset of release of quetiapine from the major component occurs within about 2 hours, for example within about 1 hour, prior to the start of a sleep period. Substantial onset of release occurs very shortly after administration, in some cases within minutes and in most cases within about 1 hour following administration. Thus time of administration should be within about 4 hours prior to the start of the sleep period, in various embodiments within about 3 hours, within about 2 hours, within about 1.5 hours or within about 1 hour prior to the start of the sleep period, or at bedtime.

Release of quetiapine from the major component is normally substantially complete within about 3 hours, in some embodiments within about 2 hours, following substantial onset of release. Release dynamics may be determined by well-known in vitro disintegration and dissolution methods, for example using a dissolution medium of GI fluid or a simulation thereof. “Substantial onset” of release will be understood to refer to a time, shortly after exposure of a dosage form or component thereof to a dissolution medium, when release of quetiapine first exceeds an inconsequential amount, for example no more than about 10% or no more than about 5%, of the total amount in the dosage form or component thereof. “Substantially complete” release will be understood to refer to a time following exposure of a dosage form or component thereof to a dissolution medium when no more than an inconsequential amount, for example no more than about 10%, or no more than about 5%, of the total amount originally present in the dosage form or component thereof, remains unreleased.

The sedative effect of the major component generally lasts no more than about 10 hours, for example no more than about 8 hours, after substantial onset of release. In some embodiments, the sedative effect is sufficient to promote sleep early in the sleep period but not to provide sedation for the whole of the sleep period. For example, in such embodiments, the sedative effect of the major component lasts no more than about 6 hours, for example no more than about 4 hours, after substantial onset of release.

Degree and duration of sedation are dose-related; a dose providing a desired degree and/or duration of sedation may be established for a particular patient by a physician without undue experimentation, based on information provided herein. Typically, the major component comprises about 100 to about 1600 mg, for example about 200 to about 1000 mg, about 200 to about 800 mg, about 300 to about 800 mg, about 300 to about 600 mg or about 300 to about 400 mg, quetiapine.

Without being bound by theory, it is believed as indicated above that the therapeutic benefits and/or enhanced side-effect profile of quetiapine when administered in accordance with dosages and methods described herein result in part from the 24-hour profile of striatal dopamine D₂ receptor occupancy provided by such administration. Striatal dopamine D₂ receptor occupancy in vivo may be estimated by known procedures including PET scanning. In some embodiments, the major component comprises an amount of quetiapine effective, upon administration to a human subject, to achieve at least about 35%, in many cases at least about 50%, occupancy by quetiapine of striatal dopamine D₂ receptors within about 4 hours, for example within about 3 hours, after administration of the major component. For example, the amount of quetiapine in the major component may be effective to achieve at least about 35%, at least about 40%, at least about 45%, at least about 50%, at least about 55%, at least about 60% or at least about 65% striatal dopamine D₂ receptor occupancy within about 4 hours, in some embodiments within about 3 hours or within about 2 hours, after administration of the major component.

It is further believed, again without being bound by theory, that striatal dopamine D₂ receptor occupancy is related to a peak plasma level, or Cₘₐₓ, of quetiapine that is transiently attained following administration of the major component, in most cases within about 4 hours, for example within about 3 hours or within about 2 hours, after administration of the major component. Cₘₐₓ may be determined by standard PK studies in human subjects. In various embodiments, the major component comprises an amount of quetiapine effective, upon administration to a human subject, to transiently attain a Cₘₐₓ sufficient to result in a clinically effective striatal dopamine D₂ receptor occupancy level, for example a Cₘₐₓ of at least about 200 ng/ml, at least about 300 ng/ml, at least about 400 ng/ml, at least about 500 ng/ml or at least about 600 ng/ml.

The transient nature of the Cₘₐₓ is believed, again without being bound by theory, to be important. It is believed, in accordance with the dosage form described herein, not to be desirable to provide the major quetiapine component in a form, for example an extended-release or pulsed-release form, that results in plasma quetiapine remaining at a high level or “plateau” for a prolonged period. Such a “plateau” of plasma quetiapine concentration may result in more continuous high-level striatal dopamine D₂ receptor occupancy than is believed to be desirable. It is believed that such occupancy should instead be allowed to fall from a peak level of at least about 35% (in many cases at least about 50%, for example at least about 55%, at least about 60% or at least about 65%) to a lower level before substantial onset of release occurs from the one or more minor quetiapine components as described below. In various embodiments that lower level is at least about 10, at least about 15, at least about 20 or at least about 25 percentage points lower than the peak level attained; in certain embodiments a striatal dopamine D₂ receptor occupancy level lower than about 50%, for example lower than about 40%, lower than about 30%, lower than about 25%, lower than about 20% or lower than about 15%, is reached prior to substantial onset of release from the one or more minor quetiapine components.

Turning now to the one or more minor quetiapine components, these may comprise a single minor component in extended-release form or a plurality of sequentially administered minor components individually in immediate-release form. Collectively the one or more minor components are present in an anxiolytic effective amount, but in an amount insufficient to cause an unacceptable degree of sedation. Time of administration and release properties are such that substantial onset of release of quetiapine from the one or more minor components occurs not earlier than about 6 hours after the start of the sleep period, i.e., not earlier than about 6 hours after administration of the major component, typically not earlier than about 7, 8, 9 or 10 hours after administration of the major component.

An “anxiolytic effect” herein comprises a reduction in agitation or excitement, i.e., a calming effect, in a subject treated with quetiapine. An anxiolytic effective amount of quetiapine is typically considerably smaller than a sedative effective amount. While sedation is desirable during a sleep period, especially during the early part of a sleep period, an anxiolytic effect is desirable during the waking period between sleep periods, and may be especially desirable shortly after awakening from a sleep period. To some extent, an anxiolytic effect may be provided as a residual effect of the major quetiapine component, but the one or more minor components supplement any such residual effect and prolong (or renew) the anxiolytic effect during the waking period.

Without being bound by theory, it is believed important according to the dosages and methods provided herein that substantial onset of release of quetiapine from the one or more minor components should not occur while plasma levels resulting from release of the major component remain high and/or while striatal dopamine D₂ receptors remain at a high level of occupancy by quetiapine. For this reason, time of administration and release properties of the one or more minor components should be such as to provide substantial onset of release therefrom not earlier than about 6 hours after the start of the sleep period (i.e., not earlier than about 6 to about 10 hours after administration of the major component).

Unlike the transient peak plasma level provided by the major component, the one or more minor components collectively provide a more sustained, although lower, plasma level. This may be accomplished by administering a plurality of immediate-release dosage forms at one or more intervals, typically of not less than about 3 hours, during the waking period, for example four doses at intervals of about 3 to about 4 hours, three doses at intervals of about 4 to about 6 hours or two doses at an interval of about 5 to about 8 hours. Alternatively, this may be accomplished by administering a single extended-release or pulsed-release dosage form early in the waking period. In yet another alternative, the one or more minor components may exhibit delayed as well as sustained or pulsed release, such that administration may take place along with the major component, for example in a single dual-release dosage form as described in greater detail hereinbelow.

As in the case of sedative effect, degree and duration of anxiolytic effect are dose-related, and a dose providing a desired degree and/or duration of anxiolytic effect may be established for a particular patient by a physician without undue experimentation, based on information provided herein. Typically, the one or more minor components collectively comprise about 10 to about 300 mg, for example about 25 to about 200 mg, about 25 to about 100 mg or about 25 to about 75 mg, in particular embodiments about 25 to about 50 mg or about 50 to about 75 mg, quetiapine.

Quetiapine is administered in a total daily amount (i.e., the sum of amounts in the major and the one or more minor components) that is therapeutically effective for the condition being treated. In most cases, a total daily dose of quetiapine administered to an adult human subject is about 50 to about 1700 mg, for example, in the case of psychiatric conditions about 100 to about 1000 mg, about 200 to about 1000 mg, about 250 to about 900 mg or about 300 to about 800 mg, in various embodiments about 300 to about 400 mg, about 300 to about 600 mg or about 400 to about 800 mg. Other doses may be found useful in particular circumstances.

In some situations the major component alone may be sufficient to provide a desired antipsychotic effect and the one or more minor components in such situations provide a complementary anxiolytic benefit during the waking period. In other situations the one or more minor components contribute to, supplement or extend the antipsychotic effect of the major component.

Although the major component must by definition provide a greater dosage amount of quetiapine than the one or more minor components, a wide range of weight ratios of these components may be used within the scope dosages and methods described herein. In most cases, a weight ratio of major to minor components of quetiapine of at least about 2:1, for example about 2:1 to about 80:1, will be found suitable, in various embodiments about 3:1 to about 60:1, about 4:1 to about 40:1, about 5:1 to about 35:1, about 6:1 to about 30:1, about 8:1 to about 20:1 or about 10:1 to about 15:1. Where a plurality of minor components are administered during a waking period, the above ratios are calculated on the total amount of quetiapine in the minor components. Without being bound by theory, it is believed that relatively low dosage levels of the one or more minor components, as reflected by a major to minor component weight ratio of at least about 4:1, may minimize re-occupation of dopamine D₂ receptors and thereby minimize risk of EPS and loss of atypicality, yet provide the desired daytime anxiolytic effect.

Illustrative major and minor component amounts for daily administration include, without limitation, 1600 mg+25 mg, 1600 mg+50 mg, 1600 mg+75 mg, 1600 mg+100 mg, 1200 mg+25 mg, 1200 mg+50 mg, 1200 mg+75 mg, 1200 mg+100 mg, 800 mg+25 mg, 800 mg+50 mg, 800 mg+75 mg, 800 mg+100 mg, 775 mg+25 mg, 750 mg+50 mg, 725 mg+75 mg, 700 mg+100 mg, 600 mg+25 mg, 600 mg+50 mg, 600 mg+75 mg, 600 mg+100 mg, 575 mg+25 mg, 550 mg+50 mg, 525 mg+75 mg, 500 mg+100 mg, 400 mg+25 mg, 400 mg+50 mg, 400 mg+75 mg, 400 mg+100 mg, 375 mg+25 mg, 350 mg+50 mg, 325 mg+75 mg, 300 mg+100 mg, 300 mg+25 mg, 300 mg+50 mg, 300 mg+75 mg, 275 mg+25 mg, 250 mg+50 mg, 225 mg+75 mg, 200 mg+25 mg, 200 mg+50 mg, 175 mg+25 mg and 150 mg+50 mg.

It is common when a patient starts a course of treatment with an antipsychotic drug such as quetiapine to titrate the daily dose from an initial low (“starting”) dose, in one to a plurality of steps, to a full dose. Thus starting and titration doses may be lower than those indicated above. Both major and minor components may be titrated together, for example maintaining the major to minor component ratio in a narrow range; or the major component only may be titrated, while the minor component remains fixed. Other titration regimes will be evident to one of skill in the art based on the disclosure herein.

Oral administration herein includes per os (p.o.), with swallowing of the drug or the dosage form containing the drug to deliver it to the GI tract. “Intraoral” (e.g., sublingual or buccal) administration that results in a substantial degree of uptake via oral mucosal surfaces as opposed to uptake from the GI tract may be used for the immediate-release major component, but is generally less suitable for the one or more minor components unless the extended-release or pulsed-release properties in the GI tract of the one or more minor components may be preserved. A “fast-melt” dosage form that disintegrates in the mouth but delivers the drug to the GI tract by swallowing may in some circumstances be used.

Where the major and minor components are provided in separate dosage forms, a suitable regimen may comprise administration of a dosage form comprising the major component prior to a sleep period (i.e., for most patients in the evening, for example at bedtime), and administration of one to a plurality of dosage forms comprising the minor component(s) after the sleep period (i.e., for most patients in the morning).

AstraZeneca recently announced (http://www.astrazeneca.com/pressrelease/5330.aspx, May 18, 2007) FDA approval of SEROQUEL XR™ extended-release quetiapine fumarate tablets, a once-daily medicine for treatment of schizophrenia in adult patients. No admission is made as to the status of this reference as prior art or otherwise with respect to the present invention. In one embodiment, a single minor quetiapine component is administered each morning in the form of a SEROQUEL XR™ tablet or a dosage form substantially bioequivalent thereto. According to this embodiment, the major quetiapine component may be administered each evening in the form of any immediate-release quetiapine formulation, including but not limited to SEROQUEL® immediate-release tablets of AstraZeneca or a dosage form substantially bioequivalent thereto.

In an alternative embodiment, the major quetiapine component is administered each evening in the form of any immediate-release quetiapine formulation, including but not limited to SEROQUEL® immediate-release tablets of AstraZeneca or a dosage form substantially bioequivalent thereto, and a plurality of minor components are administered during the day in the form of any immediate-release quetiapine formulation, including but not limited to SEROQUEL® immediate-release tablets or a dosage form substantially bioequivalent thereto.

Patient compliance or lack thereof is a well-known issue for antipsychotic medication generally, and no exception to this exists for quetiapine. The greater the number of dosages that have to be taken each day, the greater is the risk of failure of patient compliance. It is generally preferred, therefore, to administer the major and minor components according to the methods described herein in a single pharmaceutical composition, once daily at approximately the same time each day. A “single pharmaceutical composition” in the present context is not limited to a single unit dosage form, such as a single tablet or capsule, but may comprise one to a plurality of such unit dosage forms. However, each such unit dosage form comprises both the major and minor quetiapine components, in a suitable ratio as set forth above, the major component being in immediate-release form and the one or more minor components being in delayed extended-release, or delayed pulsed-release, form.

Such a dosage form useful in the present method is itself a further embodiment of the invention.

International Patent Publication No. WO 2006/081347 relates to a controlled-release composition comprising an antipsychotic agent, for example a dibenzothiazepine derivative such as quetiapine or a salt thereof. No admission is made as to the status of this reference as prior art or otherwise with respect to the present invention.

The composition of the '347 publication is said to comprise a first component that can be an immediate-release component and at least one subsequent component that can be a modified-release component. A period of time, known as “lag time”, is said to occur between release from the first component and release from the subsequent component. It is proposed that the modified release from the subsequent component may be a “delayed immediate release” or a “controlled release”, e.g., for up to 24 hours. The duration of the lag time may be 4 hours but is said to be variable through formulation alteration. It is stated that quetiapine fumarate may be “present in each component of the formulation in an amount of from about 0.1 mg to about 1 g, preferably in an amount of from about 0.1 mg to 500 mg, more preferably in an amount of from 0.5 to 60 mg, and more preferably in an amount of from 2.5 to 30 mg.”

In one embodiment, an orally deliverable pharmaceutical dosage form comprising quetiapine in a dosage amount effective for treatment of a condition for which quetiapine is indicated, and at least one pharmaceutically acceptable excipient; the quetiapine being in a form of free base and/or a pharmaceutically acceptable salt thereof, and comprising
(a) a major component in immediate-release form in a sedative effective amount, and
(b) one or more minor components in delayed extended-release or delayed pulsed-release form, collectively in an anxiolytic effective amount insufficient to cause an unacceptable degree of sedation;
   wherein, upon exposure of the dosage form to GI fluid or an in vitro medium that simulates GI fluid, substantial onset of release of quetiapine from the one or more minor components exhibits a delay of about 6 to about 24 hours after initiation of said exposure is provided.

The definition of “major” and “minor” is as provided hereinabove, but typically the major component comprises at least about 67%, more typically at least about 80%, by weight of all the quetiapine present in the dosage form, i.e., the weight ratio of major to minor components of quetiapine is typically at least about 2:1, more typically at least about 4:1. For example, ratios of about 2:1 to about 80:1 may be found suitable, in various embodiments about 3:1 to about 60:1, about 4:1 to about 40:1, about 5:1 to about 35:1, about 6:1 to about 30:1, about 8:1 to about 20:1 or about 10:1 to about 15:1.

Illustrative major and minor component amounts in the dosage form include, without limitation, 1600 mg+25 mg, 1600 mg+50 mg, 1600 mg+75 mg, 1600 mg+100 mg, 1200 mg+25 mg, 1200 mg+50 mg, 1200 mg+75 mg, 1200 mg+100 mg, 800 mg+25 mg, 800 mg+50 mg, 800 mg+75 mg, 800 mg+100 mg, 775 mg+25 mg, 750 mg+50 mg, 725 mg+75 mg, 700 mg+100 mg, 600 mg+25 mg, 600 mg+50 mg, 600 mg+75 mg, 600 mg+100 mg, 575 mg+25 mg, 550 mg+50 mg, 525 mg+75 mg, 500 mg+100 mg, 400 mg+25 mg, 400 mg+50 mg, 400 mg+75 mg, 400 mg+100 mg, 375 mg+25 mg, 350 mg+50 mg, 325 mg+75 mg, 300 mg+100 mg, 300 mg+25 mg, 300 mg+50 mg, 300 mg+75 mg, 275 mg+25 mg, 250 mg+50 mg, 225 mg+75 mg, 200 mg+25 mg, 200 mg+50 mg, 175 mg+25 mg and 150 mg+50 mg.

The form in which the one or more minor components are present in the dosage form may be a delayed pulsed-release form or a delayed extended-release form. A simple delayed-release profile, in which a bolus of drug akin to an immediate-release bolus is released in a short period of time (a so-called “delayed immediate-release” profile) is not desired. It is believed, without being bound by theory, that a single minor component exhibiting “delayed immediate release” will either (if present in a low dosage amount) provide too transient an anxiolytic effect or (if present in a sufficient dosage amount to provide a more lasting anxiolytic effect) cause unwanted daytime sedation.

A dosage form of the present embodiment is adapted for once-daily oral administration within about 4 hours prior to the start of a sleep period, for example within about 3 hours, within about 2 hours, within about 1.5 hours or within about 1 hour prior to the start of the sleep period, or at bedtime.

In one embodiment, a dosage form as described herein may be defined by the PK profile it exhibits when orally administered to a human subject or a plurality of such subjects according to a standard PK protocol. It is generally preferred to define the dosage form in terms of a steady-state PK profile reached by administration at a therapeutically effective dose once daily for several days, usually at least about 3 days, optionally following a period of dosage titration to reach such therapeutically effective dose. It is emphasized that receptor occupancy by quetiapine, rather than blood plasma levels of quetiapine, is believed to be the key to the benefits afforded by the embodiments described herein; however, as explained hereinabove, maximum plasma levels and alternation of high and low plasma levels are important in providing appropriate levels and intermittency of receptor occupancy.

According to this embodiment, the steady-state PK profile of the dosage form exhibits at least two peaks, Cₘₐₓ₁ and Cₘₐₓ₂, and at least two troughs, Cₘᵢₙ₁ and Cₘᵢₙ₂. The relative size and timing of these peaks and troughs is dependent upon, among other things, the amount of quetiapine present in each of the major and minor components, the delay time between substantial onset of release of the major component and substantial onset of release of the minor component(s), and the particular release profile of the minor component(s)—in particular whether a delayed pulsed-release or delayed extended-release profile is selected. It will be understood that the word “peak” in reference to Cₘₐₓ₂ is used for convenience and does not necessarily imply existence of a sharp peak.

Cₘₐₓ₁ is the higher of the at least two peaks and is provided by the major component. As the major component exhibits immediate-release properties, Cₘₐₓ₁ generally occurs at a time (Tₘₐₓ₁) that is relatively shortly after administration. Tₘₐₓ₁ is normally less than about 4 hours, for example about 0.5 to about 3 hours, about 0.7 to about 2.5 hours or about 1 to about 2 hours after administration. The value of Cₘₐₓ₁ is strongly dose-dependent. In various embodiments the amount of quetiapine in the major component of a dosage form is sufficient to provide a Cₘₐₓ₁ of at least about 200 ng/ml, for example at least about 250 ng/ml, at least about 300 ng/ml, at least about 350 ng/ml, at least about 400 ng/ml or at least about 450 ng/ml.

Cₘᵢₙ₁ represents the trough, resulting from natural elimination of the major component quetiapine from plasma, reached immediately prior to substantial onset of release of the one or more minor components. Importantly, Cₘᵢₙ₁ occurs at a time (Tₘᵢₙ₁) that is not less than about 6 hours after the start of the sleep period following administration; depending on the precise timing of administration, Tₘᵢₙ₁ may be not less than about 6 hours, not less than about 7 hours, not less than about 8 hours or not less than about 9 hours after administration. In various embodiments Tₘᵢₙ₁ may be up to about 18 hours, for example up to about 16 hours, up to about 14 hours or up to about 12 hours after administration. How low a plasma level Cₘᵢₙ₁ may be achieved depends on the value of Cₘₐₓ₁ and the time elapsed between Tₘₐₓ₁ and Tₘᵢₙ₁; in some embodiments Cₘᵢₙ₁ is less than about 150 ng/ml, for example less than about 100 ng/ml, less than about 75 ng/ml or less than about 50 ng/ml.

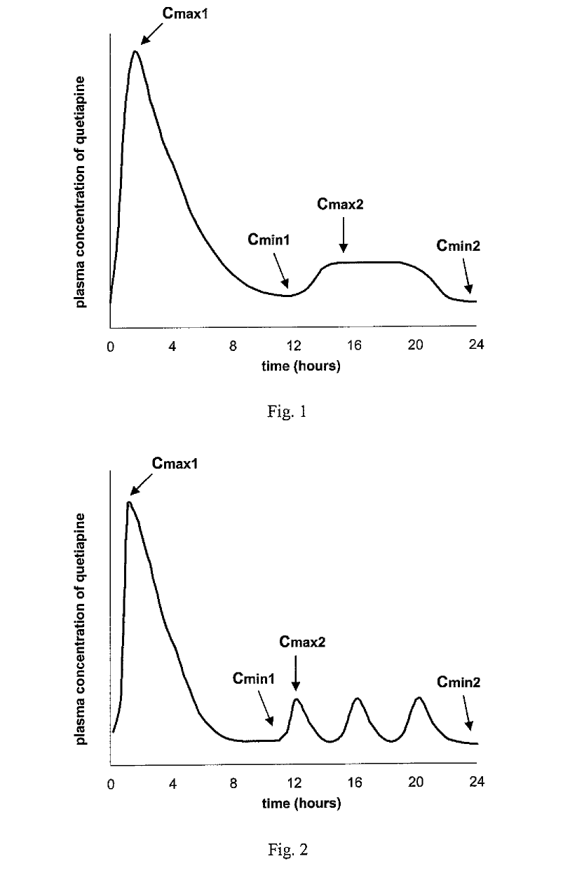

Cₘₐₓ₂ is substantially lower than Cₘₐₓ₁. Where the minor component is in extended-release form, Cₘₐₓ₂ represents the highest point on a relatively flat but low plateau of plasma quetiapine concentration as illustrated schematically in FIG. 1. Where the minor component is in pulsed-release form, Cₘₐₓ₂ represents the highest of a plurality of low peaks, typically the first of such peaks, as illustrated schematically in FIG. 2. Cₘₐₓ₂ is generally not greater than about 200 ng/ml, for example not greater than about 150 ng/ml, not greater than about 120 ng/ml or not greater than about 100 ng/ml.

Cₘᵢₙ₂ represents the trough, resulting from natural elimination of both major and minor components, reached immediately prior to administration of the next daily dose.

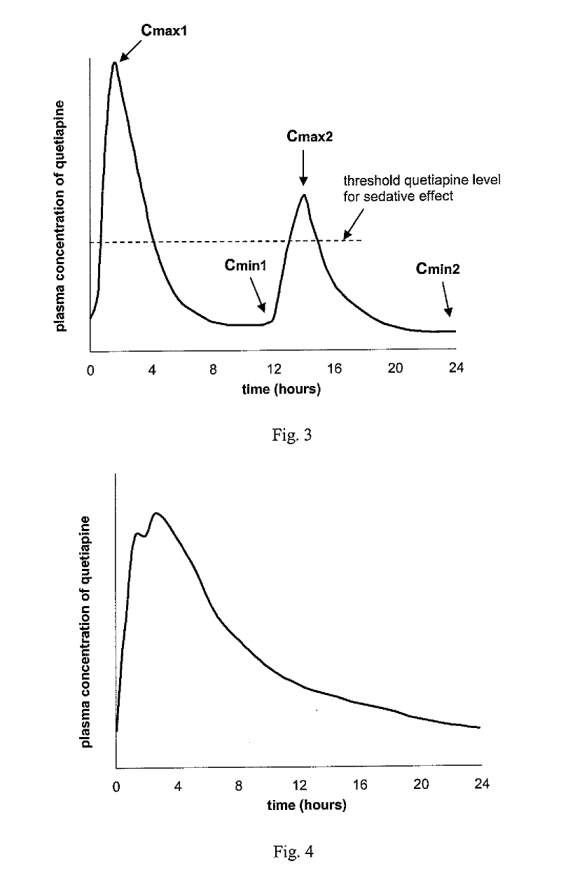

FIGS. 3 and 4 schematically illustrate the PK profile of two comparative once-daily dosage forms that fail to meet the requirements of the present invention. In FIG. 3, the minor component is in “delayed immediate-release” form, and release of the minor component all at once results in a Cₘₐₓ₂ that is above a threshold causing sedation. In FIG. 4, there is only one extended-release component, which results in a sustained high plasma concentration that is, as described above, believed conducive to development of EPS and loss of atypicality of quetiapine.

Thus the present invention does not seek to maintain a high blood plasma level over a prolonged period. The trough represented by Cₘᵢₙ₁ is very important, as is the relatively low value of Cₘₐₓ₂. In some embodiments, the ratio of Cₘₐₓ₁ to Cₘᵢₙ₁ is at least about 2:1, for example at least about 3:1, at least about 4:1 or at least about 5:1, and may be as much as 10:1, for example as much as 20:1 or even greater. In some embodiments, the ratio of Cₘₐₓ₁ to Cₘₐₓ₂ is at least about 1.5:1, for example at least about 2:1, at least about 3:1 or at least about 4:1, and may be as much as 8:1 or even greater.

The pharmaceutical dosage form described herein may be solid, semi-solid or liquid but typically is a discrete solid dosage form such as a tablet, caplet, pill, pellet, hard or soft capsule, lozenge or troche. One or more such dosage forms may contain sufficient quetiapine to provide a single daily dose. In one embodiment, the entire daily dose of quetiapine, including both major and minor components as described above, is contained in a single dosage form.

Any immediate-release formulation technology may be used for the major component of a dosage form as described herein. Such technology typically employs conventional pharmaceutically acceptable excipients including, in the case of a solid dosage form such as a tablet or capsule, one or more of a diluent, a binder, a disintegrant, a wetting agent and/or an anti-adherent (e.g., a lubricant and/or glidant).

Any formulation technology that provides modified release, including but not limited to extended release, delayed release, sustained release, controlled release, pulsed release, and so forth, may be used for the minor component of a dosage form as described herein.

In some embodiments, the major and minor components may be more or less intimately co-mixed or blended (e.g., within individual granules that are subsequently encapsulated or compressed into tablets).

In other embodiments, the major and minor components may form spatially distinct zones of the dosage form. Illustratively, the zones may be disposed as follows:
(a) as two or more layers of a bilayer or multilayer tablet;
(b) as two or more compartments of a multi-compartment capsule;
(c) as two or more pre-compressed or pre-molded tablets embedded within a single larger dosage form;
(d) as a mantle surrounding and substantially enclosing a core, with or without an enteric coating layer immediately surrounding the core (in this embodiment the major component is generally in the mantle and the one or more minor components in the core);
(e) as a plurality of particles compressed or molded into a single tablet, wherein the term “particles” embraces granules, beads, individual particles in a multiparticulate formulation, etc.;
(f) as a plurality of beads or microtablets, at least a fraction of which have a release-modifying coating; or
(g) as a plurality of beads or microtablets, at least a fraction of which individually comprise a mantle surrounding and substantially enclosing a core.

Illustratively, delayed release is provided by investing the one or more minor components with a coating or barrier layer that substantially prevents release for the desired period after administration, and then, by a process of erosion, dissolution or disintegration, permits substantial onset of release. The coating may be enteric, i.e., designed to erode, dissolve or disintegrate upon reaching a particular zone of the GI tract, for example through a pH response. Alternatively, rupture of the coating may be simply time-dependent.

In one embodiment, modified release is provided by a plurality of populations of beads or microtablets having individual barrier layers, wherein release time is relatively constant within populations but differs between populations. In another embodiment, modified release is provided by a plurality of beads or microtablets having individual barrier layers, wherein release time varies more or less continuously among beads or microtablets. Formulations of these embodiments are referred to herein as “coated bead formulations.”

Coated bead formulations may be prepared as a component of capsules or of tablets. The coating or barrier layer may be designed to achieve controlled dissolution and/or diffusion of the drug from the dosage form. Several functional excipients may be used for this purpose, e.g., waxes, fatty materials, polymers, natural gums, synthetic gums, semi-synthetic gums, and so forth. The coating or barrier layer may comprise one or more polymers, and typically includes a polymer of low solubility in water. Suitable polymers for use in barrier layers of modified release pharmaceutical compositions are known to those of skill in the art, and include, but are not limited to, cellulose ethers, cellulose esters, polymers and copolymers of acrylic acid and methacrylic acid and esters, poly vinyl derivatives, polyethylene glycol, hydrogenated castor oil, hydrogenated vegetable oil, shellac, zein, natural and synthetic waxes, fatty acids, fatty alcohols, and combinations thereof. Some non-limiting examples include hydroxypropylcellulose (HPC), hydroxyethylcellulose, methylhydroxyethylcellulose, hydroxypropylmethylcellulose (HPMC or hypromellose), methylcellulose, ethylcellulose, cellulose acetate, sodium carboxymethylcellulose (carmellose sodium), polymers and copolymers of acrylic acid and methacrylic acid and esters thereof, polyvinylpyrrolidone (povidone) and polyethylene glycol (PEG).

In yet another embodiment, modified release is provided by distributing the drug in a matrix comprising one or more swellable or erodible polymers. The drug-polymer matrix may be enclosed within a delayed-release coating. Suitable polymers for use in modified release matrix compositions are known to those of skill in the art. One of the most widely used is HPMC.

In general, excipients capable of slowing quetiapine release may be hydrophilic or hydrophobic, and include, but are not limited to, HPMC, HPC, carboxyvinyl polymers, polyvinyl alcohol, glucans, scleroglucans, mannans, xanthans, carboxymethylcellulose and its derivatives, alkylcelluloses such as methylcellulose and ethylcellulose, acrylic and methacrylic acid polymers and copolymers, shellac, zein, hydrogenated castor oil, hydrogenated vegetable oil, natural and synthetic waxes, fatty acids, fatty alcohols and combinations thereof, and so forth.

One method described herein optionally further comprises monitoring of therapeutic, e.g., antipsychotic, antidepressant, sedative or anxiolytic efficacy and incidence of adverse effects at suitable intervals, to permit adjustment of dose and/or change of the prescription to a dosage form having different release characteristics.

Optionally, a method as described herein further comprises administering to the subject one or more additional pharmaceutical agents. An additional pharmaceutical agent may be administered, for example, in combination or adjunctive therapy with the quetiapine. Any pharmaceutical agent appropriate to the particular condition being treated may be used.

In one embodiment quetiapine is administered as described herein, in adjunctive or combination therapy with one or more additional agents selected from mood stabilizers, anticonvulsants, antidepressants, anti-anxiety agents, tranquilizers, antipsychotics and sleep aids.

Suitable mood stabilizers and anticonvulsants illustratively and without limitation include lithium, valproic acid, carbamazepine, lamotrigine, gabapentin, topiramate, oxcarbazepine, olanzapine, verapamil, risperidone, aripiprazole, ziprasidone and levetiracetam.

Suitable antidepressants illustratively and without limitation include fluoxetine, fluvoxamine, paroxetine, sertraline, venlafaxine, nefazodone, bupropion, mirtazapine, phenelzine, tranylcypromine, amitriptyline, desipramine, imipramine, nortriptyline, isocarboxazid, citalopram, reboxetine, clomipramine, doxepin, trazodone and escitalopram.

Suitable anti-anxiety drugs and tranquilizers illustratively and without limitation include buspirone, hydrazine and benzodiazepines including those listed hereinbelow.

Suitable antipsychotics illustratively and without limitation include clozapine, olanzapine, risperidone, chlorpromazine, haloperidol, perphenazine, fluphenzine, sertindole, ziprasidone, aripiprazole, amisulpride, zotepine, trifluoperazine, thiothixene, thioridazine, flupentixol and cyamemazine.

Suitable sleep aids illustratively and without limitation include zolpidem, chloral hydrate, diphenhydramine, doxylamine, eszopiclone, hydroxyzine, lorazepam, ramelteon, triazolam, zaleplon, zopiclone, flurazepam, quazepam, estazolam, modafinil, zaleplon and trazodone.

In one embodiment, quetiapine is administered as described herein, in adjunctive or combination therapy with one or more benzodiazepines, for example alprazolam, bromazepam, diazepam, lorazepam, clorazepam, clonazepam, temazepam, oxazepam, flunitrazepam, triazolam, chlordiazepoxide, flurepam, estazolam, nitrazepam, meprobamate, prazepam, flurazepam, clorazepate, nordazepam, midazolam and/or quazepam.

All patents and publications cited herein are incorporated by reference into this application in their entirety.

The words “comprise”, “comprises”, and “comprising” are to be interpreted inclusively rather than exclusively.

What is claimed is:

1. A method for treating a medical condition for which quetiapine is indicated in a subject, comprising orally administering to the subject quetiapine or a pharmaceutically acceptable salt thereof in a daily dosage amount effective to treat said condition; wherein the quetiapine or salt thereof is administered in one to a plurality of dosage forms collectively comprising
(a) a major quetiapine component in immediate-release form in a sedative effective amount, administered not earlier than about 4 hours prior to the start of a sleep period; and
(b) either (i) a minor quetiapine component in extended-release, delayed extended-release or delayed pulsed-release form, wherein time of administration and release properties of the minor component provide substantial onset of release of quetiapine therefrom not earlier than about 6 hours after the start of the sleep period, or (ii) a plurality of minor quetiapine components in immediate-release form, administered sequentially during a waking period following the sleep period but not earlier than about 6 hours after the start of the sleep period; said minor component or components collectively being administered in an anxiolytic effective amount insufficient to cause an unacceptable degree of sedation.

2. The method of claim 1, wherein the medical condition comprises a psychiatric condition.

3. The method of claim 2, wherein the psychiatric condition comprises schizophrenia, bipolar mania and/or a depressive phase of a bipolar condition.

4. The method of claim 1, wherein the medical condition comprises a sleep disorder.

5. The method of claim 1, wherein the minor component or components are provided by an extended-release dosage form administered once, or a plurality of immediate-release dosage forms administered sequentially, during a waking period following the sleep period.

6. The method of claim 5, wherein the minor component or components are provided by an extended-release dosage form administered once.

7. The method of claim 1, wherein all of said quetiapine components are administered not earlier than about 4 hours prior to the sleep period in one to a plurality of dosage forms, each comprising said major component and said one or more minor components in delayed extended-release or delayed pulsed-release form; and wherein, upon exposure of any one of said dosage forms to GI fluid or an in vitro medium that simulates GI fluid, substantial onset of release of quetiapine from the one or more minor components exhibits a delay of at least about 6 hours after initiation of said exposure.

8. The method of claim 7, wherein administration occurs not earlier than about 1 hour prior to a sleep period.

9. The method of claim 1, wherein the daily quetiapine dosage amount is initially substantially lower than a maintenance dose and is titrated upward in one or more increments until the maintenance dose is reached.

10. The method of claim 1, comprising daily administration in total of about 25 mg to about 1700 mg quetiapine.

11. The method of claim 1, comprising daily administration in total of about 100 mg to about 1000 mg quetiapine.

12. The method of claim 1, comprising daily administration in total of about 200 mg to about 800 mg quetiapine.

13. The method of claim 1, wherein the major component comprises about 100 mg to about 1600 mg quetiapine.

14. The method of claim 1, wherein the major component comprises about 200 mg to about 800 mg quetiapine.

15. The method of claim 1, wherein the minor component(s) collectively comprise about 10 mg to about 300 mg quetiapine.

16. The method of claim 1, wherein the minor component(s) collectively comprise about 25 mg to about 200 mg quetiapine.

17. The method of claim 1, wherein the major component and collectively the minor component(s) are present in a quetiapine weight ratio of about 2:1 to about 40:1.

18. The method of claim 1, wherein the major component and collectively the minor component(s) are present in a quetiapine weight ratio of about 2:1 to about 80:1.

19. The method of claim 1, further comprising administering to the subject in combination or adjunctive therapy one or more additional agents selected from the group consisting of mood stabilizers, anticonvulsants, antidepressants, anti-anxiety agents, tranquilizers, antipsychotics, sleep aids and combinations thereof.

20. A pharmaceutical dosage form useful in the method of claim 7.

21. An orally deliverable pharmaceutical dosage form comprising quetiapine in a dosage amount effective for treatment of a condition for which quetiapine is indicated, and at least one pharmaceutically acceptable excipient; the quetiapine being in a form of free base and/or a pharmaceutically acceptable salt thereof, and comprising
(a) a major component in immediate-release form in a sedative effective amount, and
(b) one or more minor components in delayed extended-release or delayed pulsed-release form, collectively in an anxiolytic effective amount insufficient to cause an unacceptable degree of sedation;
wherein, upon exposure of the dosage form to GI fluid or an in vitro medium that simulates GI fluid, substantial onset of release of quetiapine from the one or more minor components exhibits a delay of about 6 to about 24 hours after initiation of said exposure.

22. The dosage form of claim 21, wherein release of quetiapine from the major component is substantially complete within about 4 hours after initiation of said exposure.

23. The dosage form of claim 21, wherein release of quetiapine from the major component is substantially complete within about 2 hours after initiation of said exposure.

24. The dosage form of claim 21, wherein said quetiapine comprises a minor component in delayed extended-release form.

25. The dosage form of claim 21, wherein said delay is of about 8 to about 18 hours.

26. The dosage form of claim 21, wherein said delay is of about 10 to about 14 hours.

27. The dosage form of claim 21, wherein the quetiapine is present in whole or part as one or more pharmaceutically acceptable salts selected from the group consisting of hydrochloride, hydrobromide, sulfate, sulfamate, phosphate, nitrate, acetate, trifluoroacetate, propionate, oxalate, malonate, succinate, glutarate, glycolate, stearate, lactate, malate, tartrate, citrate, ascorbate, pamoate, maleate, hydroxymaleate, fumarate, phenylacetate, arginate, asparginate, glutamate, benzoate, salicylate, mesylate, esylate, besylate, sulfanilate, 2-acetoxybenzoate, toluenesulfonate, methanesulfonate, ethanedisulfonate, oxalate and isethionate salts and combinations thereof.

28. The dosage form of claim 21, wherein the quetiapine is in the form of a fumarate salt.

29. The dosage form of claim 21, comprising in total about 25 mg to about 1700 mg quetiapine.

30. The dosage form of claim 21, comprising in total about 200 mg to about 800 mg quetiapine.

31. The dosage form of claim 21, wherein the major component comprises about 100 mg to about 1600 mg quetiapine.

32. The dosage form of claim 21, wherein the major component comprises about 200 mg to about 800 mg quetiapine.

33. The dosage form of claim 21, wherein the one or more minor components collectively comprise about 10 mg to about 300 mg quetiapine.

34. The dosage form of claim 21, wherein the one or more minor components collectively comprise about 25 mg to about 200 mg quetiapine.

35. The dosage form of claim 21, wherein the major component and collectively the minor component(s) are present in a quetiapine weight ratio of about 2:1 to about 80:1.

36. The dosage form of claim 21, wherein the major component and collectively the minor component(s) are present in a quetiapine weight ratio of about 4:1 to about 40:1.

37. The dosage form of claim 21, wherein, when one to a plurality of such dosage forms are administered orally once daily to a subject, the major component provides greater than about 35% occupancy by quetiapine of striatal dopamine D₂ receptors within about 4 hours after administration, such occupancy subsequently falling below about 35%; and the one or more minor components upon release thereof are collectively ineffective to restore such occupancy to a level above about 35%.

38. The dosage form of claim 21, wherein, when one to a plurality of such dosage forms are administered orally once daily to a subject, the major component provides greater than about 50% occupancy by quetiapine of striatal dopamine D₂ receptors within about 4 hours after administration, such occupancy subsequently falling below about 50%; and the one or more minor components upon release thereof are collectively ineffective to restore such occupancy to a level above about 50%.

39. The dosage form of claim 38, wherein the major component provides greater than about 60% occupancy by quetiapine of striatal dopamine D₂ receptors within about 4 hours after administration.

40. The dosage form of claim 38, wherein, after said provision of greater than about 50% occupancy by the major component, such occupancy subsequently falls below about 40%.

41. The dosage form of claim 38, wherein, after said provision of greater than about 50% occupancy by the major component, such occupancy subsequently falls below about 30%.

42. The dosage form of claim 21, wherein, upon once-daily oral administration to a human subject, at least about 50% occupancy of striatal dopamine D₂ receptors by quetiapine is achieved within about 4 hours following administration; and substantial onset of release of quetiapine from the one or more minor components occurs not before occupancy of striatal dopamine D₂ receptors by quetiapine has fallen below about 50%.

43. The dosage form of claim 41, wherein, upon said administration, at least about 60% occupancy of striatal dopamine D₂ receptors by quetiapine is achieved within about 4 hours following administration; and substantial onset of release of quetiapine from the one or more minor components occurs not before occupancy of striatal dopamine D₂ receptors by quetiapine has fallen below about 40%.

44. The dosage form of claim 43, wherein substantial onset of release of quetiapine from the one or more minor components occurs not before occupancy of striatal dopamine D₂ receptors by quetiapine has fallen below about 30%.

45. The dosage form of claim 21, wherein the major component comprises an amount of quetiapine effective, upon once-daily oral administration of the dosage form to a human subject, to transiently attain a peak quetiapine blood level sufficient to initiate striatal dopamine D₂ receptor occupancy by quetiapine at a level providing an antipsychotic effect.

46. The dosage form of claim 21, wherein the major component comprises an amount of quetiapine effective, upon once-daily oral administration of the dosage form to a human subject, to transiently attain a peak quetiapine blood level of at least about 300 ng/ml.

47. The dosage form of claim 21, wherein the major component comprises an amount of quetiapine effective, upon once-daily oral administration of the dosage form to a human subject, to transiently attain a peak quetiapine blood level of at least about 400 ng/ml.

48. The dosage form of claim 21, wherein the major component comprises an amount of quetiapine effective, upon once-daily oral administration of the dosage form to a human subject, to transiently attain a peak quetiapine blood level of at least about 600 ng/ml.

49. The dosage form of claim 21, in a form of a tablet or capsule.

50. The dosage form of claim 49, wherein the major and minor components are spatially segregated.

51. The dosage form of claim 50, wherein the spatial segregation of major and minor components is
(a) as two or more layers of a bilayer or multilayer tablet;
(b) as two or more compartments of a multi-compartment capsule;
(c) as two or more pre-compressed or pre-molded tablets embedded within a single larger dosage form;
(d) as a mantle surrounding and substantially enclosing a core, with or without an enteric coating layer immediately surrounding the core;
(e) as a plurality of particles compressed or molded into a single tablet;
(f) as a plurality of beads or microtablets, at least a fraction of which have a release-modifying coating; or
(g) as a plurality of beads or microtablets, at least a fraction of which individually comprise a mantle surrounding and substantially enclosing a core.

52. An orally deliverable pharmaceutical dosage form comprising quetiapine, in a form of free base and/or a pharmaceutically acceptable salt thereof, and at least one pharmaceutically acceptable excipient; the dosage form upon once-daily administration exhibiting a steady-state PK profile having at least two peaks, Cₘₐₓ₁ and Cₘₐₓ₂, and a trough, Cₘᵢₙ₁, therebetween; wherein
(a) Cₘₐₓ₁ is a transient peak occurring less than about 4 hours after administration and is sufficient to initiate occupancy by quetiapine of striatal dopamine D₂ receptors at a level greater than about 50%, which level subsequently falls below about 50%;
(b) Cₘᵢₙ₁ occurs between about 6 hours and about 18 hours after administration;
(c) the ratio of Cₘₐₓ₁ to Cₘᵢₙ₁ is at least about 2:1; and
(d) the ratio of Cₘₐₓ₁ to Cₘₐₓ₂ is at least about 1.5:1.

53. The dosage form of claim 52, wherein
(a) Cₘₐₓ₁ occurs less than about 2 hours after administration;
(b) Cₘᵢₙ₁ occurs between about 8 hours and about 14 hours after administration;
(c) the ratio of Cₘₐₓ₁ to Cₘᵢₙ₁ is at least about 4:1; and
(d) the ratio of Cₘₐₓ₁ to Cₘₐₓ₂ is at least about 3:1.

54. The dosage form of claim 53, wherein the ratio of Cₘₐₓ₁ to Cₘᵢₙ₁ is at least about 5:1, and the ratio of Cₘₐₓ₁ to Cₘₐₓ₂ is at least about 4:1.
